(19) United States
(12) Patent Application Publication
(10) Pub. No.: US 2026/0285323 A1
(43) Pub. Date: Sep. 24, 2026

(54) IN VEHICLE STRESS REDUCTION

(71) Applicant: GM Global Technology Operations LLC, Detroit, MI (US)

(72) Inventors: Yuval Zak, Gedera (IL); Andrea Forgacs Braunshtain, Netanya (IL); Gershon Celniker, Netanya (IL); Ron Hecht, Raanana (IL)

(21) Appl. No.: 19/083,934

(22) Filed: Mar. 19, 2025

(51) Int. Cl. B60W 40/08; A61B 5/16; A61M 21/00

(52) U.S. Cl. CPC B60W 40/08; A61B 5/163; A61M 21/00; A61M 2021/0088; B60W 2040/0872

(57) ABSTRACT

A vehicle including a first set of sensors configured to monitor an operator of the vehicle. A second set of sensors is configured to monitor a scene in which the vehicle is operating. A controller in the vehicle includes a driving condition module and an operator state module. The driving condition module determines a conduciveness for a guided stress reduction exercise using the second set of sensors. The operator state module determines a stress level of the operator based on the first set of sensors. The controller includes a memory storing instructions for causing the controller to identify a stress level of the operator, identify a conduciveness of the scene to a guided stress reduction exercise, and initiating a guided stress reduction exercise in response to the stress level of the operator exceeding a threshold and the scene being conducive to performing a guided stress reduction exercise.

### INTRODUCTION

The subject disclosure relates to stress reduction systems, and more particularly to vehicle systems for identifying and engaging opportunities for stress reduction within a vehicle.

Day to day stress significantly impacts both physical and mental health and contributes to a range of chronic conditions such as cardiovascular disease, anxiety, and depression. The physiological response to stress involves release of hormones like cortisol and adrenaline. When persistently elevated (e.g., when a person is under stress for an extended period of time), such hormones can lead to detrimental health outcomes including high blood pressure, impaired immune function, and metabolic disorders.

Within the context of vehicle operations, stress can make vehicle operation more difficult by reducing cognitive functions and increasing reaction times. This in turn can lead to increased instances of unintended vehicle operations. Furthermore, stress-induced distractions and aggressive driving behaviors are common in some vehicle operators.

One effective method to mitigate stress is the use of controlled breathing exercises. Techniques such as diaphragmatic breathing and mindfulness meditation activate the parasympathetic nervous system, promoting relaxation and reducing physiological markers of stress. Regular practice of these techniques improves emotional regulation and overall resilience to stress.

Some systems have attempted to encourage users to engage in breathing sessions or other stress relief using timed reminders. However, the timed reminders can get lost in day to day notifications, neglected due to poor timing, and/or can occur while the user is performing an activity (e.g. driving a vehicle) during which the user is unable to perform the stress relief exercise.

Accordingly, it is desirable to provide a process for identifying and engaging opportunities to reduce stress during the operation of a vehicle.

### SUMMARY

In one exemplary embodiment a vehicle including a first set of sensors configured to monitor an operator of the vehicle. A second set of sensors is configured to monitor a scene in which the vehicle is operating. A controller in the vehicle includes a driving condition module and an operator state module. The driving condition module is configured to determine a conduciveness for a guided stress reduction exercise using the second set of sensors. The operator state module is configured to determine a stress level of the operator based at least in part on the first set of sensors. The controller includes a memory storing instructions for causing the controller to perform operations including identifying a stress level of the operator using the operator state module, identifying a conduciveness of the scene to a guided stress reduction exercise using the driving condition module, and initiating a guided stress reduction exercise in response to the stress level of the operator exceeding a threshold and the scene being conducive to performing a guided stress reduction exercise.

In addition to one or more of the features described herein the guided stress reduction exercise is a guided breathing exercise.

In addition to one or more of the features described herein the first set of sensors includes at least one camera defining a field of view including a face of the operator, and wherein the operator state module includes a gaze based driver monitoring system.

In addition to one or more of the features described herein identifying the stress level of the operator comprises identifying a gaze pattern corresponding to an elevated stress level using the gaze based driver monitoring system.

In addition to one or more of the features described herein the first set of sensors includes at least one physiological sensor disposed on the operator.

In addition to one or more of the features described herein identifying the stress level of the operator includes correlating the data in the first set of sensors as a set of time series machine learning features and providing the time series machine learning features to an input layer of a machine learning system, and wherein the machine learning system is configured to output a stress level of the operator.

In addition to one or more of the features described herein the controller includes a wireless data connection, and wherein the machine learning system is retrieved from a remote data storage responsive to the controller initiating the operations.

In addition to one or more of the features described herein the operations further include monitoring performance of the guided stress reduction exercise by the operator and assigning a score to the performance.

In addition to one or more of the features described herein the controller further includes and least one gamification feature configured to incentivize the operator increasing the score of the performance.

In addition to one or more of the features described herein the operations further include interrupting a guided exercise in response to determining that the scene is no longer conducive to performance of the guided exercise.

In another exemplary embodiment a method for reducing stress in vehicle operators includes identifying, using an operator state module, a stress level of the operator based at least in part on a gaze pattern of an operator of a vehicle. The method identifies a conduciveness to a guided stress reduction exercise of a scene in which a vehicle is operating using a driving condition module. A guided stress reduction exercise is initiated in response to the stress level of the operator exceeding a threshold and the scene being conducive to performing a guided stress reduction exercise.

In addition to one or more of the features described herein initiating the guided stress reduction exercise comprises providing a notification to the operator indicating elevated stress levels and suggesting the guided stress reduction exercise.

In addition to one or more of the features described herein initiating the guided stress reduction exercise further comprises displaying the guided stress reduction exercise on an infotainment screen of the vehicle.

In addition to one or more of the features described herein initiating the guided stress reduction exercise further comprises monitoring a performance of the guided stress reduction exercise by the vehicle operator.

In addition to one or more of the features described herein, the method further includes applying a score to the performance and ranking the performance using a gamification system.

In addition to one or more of the features described herein the operator state module includes a machine learning system configured to receive a set of inputs from a plurality of vehicle sensors including a driver monitoring camera, define machine learning features based on the set of inputs, and generate a stress level of the vehicle operator based on the defined machine learning features.

In addition to one or more of the features described herein the operator state module further includes a noise filtering process configured to filter the machine learning features using a time series smoothing process.

In addition to one or more of the features described herein the machine learning system a stateless model, with only a most recent output being stored between outputs.

In addition to one or more of the features described herein further includes receiving physiological data of the operator from at least one physiological sensor disposed on the operator, and wherein identifying the stress level of the operator is based at least in part on the physiological data.

In addition to one or more of the features described herein initiating guided stress reduction exercise further includes continuously monitoring the conduciveness of engaging in the guided stress reduction exercise and responding to a change from conducive to non-conducive by interrupting the guided stress reduction exercise.

The above features and advantages, and other features and advantages of the disclosure are readily apparent from the following detailed description when taken in connection with the accompanying drawings.

### BRIEF DESCRIPTION OF THE DRAWINGS

Other features, advantages and details appear, by way of example only, in the following detailed description, the detailed description referring to the drawings in which:

FIG. 1 is a top down view of a vehicle according to some embodiments;

FIG. 2 is a flowchart illustrating a process for identifying stress relief opportunities and engaging stress relief exercises;

FIG. 3 is a schematic illustration of a machine learning architecture for identifying a stress condition of a vehicle operator; and

FIG. 4 is an exemplary scene in which a vehicle operator is able to engage in a stress reduction exercise.

### DETAILED DESCRIPTION

The following description is merely exemplary in nature and is not intended to limit the present disclosure, its application or uses. As used herein, the term module refers to processing circuitry that may include an application specific integrated circuit (ASIC), an electronic circuit, a processor (shared, dedicated, or group) and memory that executes one or more software or firmware programs, a combinational logic circuit, and/or other suitable components that provide the described functionality.

As used herein, the term controller refers to a system or component including at least a processor and a memory, with the system or component being configured to perform or cause to be performed at least one operation. The system can be a dedicated controller including a single purpose processor and memory, a general controller including one or more modules for performing the operation, a distributed system including multiple components in communication with each other and configured to control the operation, or any similar system.

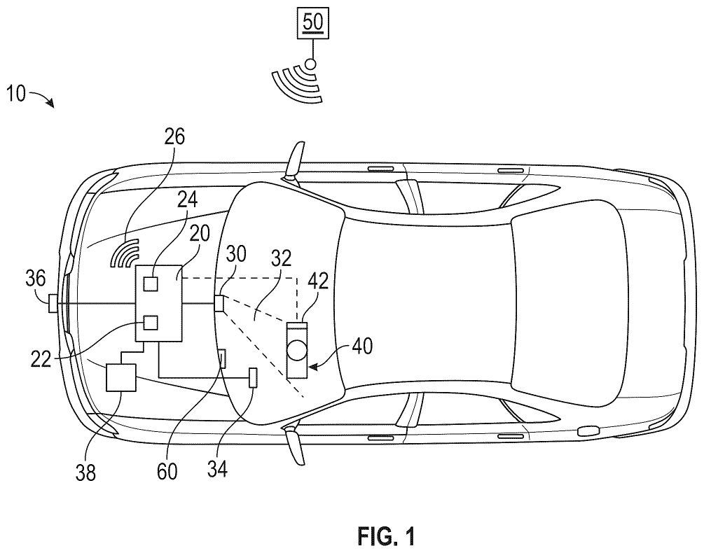

In accordance with an exemplary embodiment, FIG. 1 illustrates a vehicle 10 including a controller 20. The controller 20 is in communication with an image sensor 30. The image sensor 30 defines a field of view 32 including a head or face of a vehicle operator 40. In some examples, the image sensor 30 may be a red-green-blue (RGB) camera or infra-red (IR) camera. In other examples, any similar sensor for capturing images of the vehicle operator 40 may be used.

In some examples, the vehicle 10 also includes at least one physiological sensor 34 in communication with the controller 20. The physiological sensor 34 measures one or more physiological state (e.g. pulse rate) of the vehicle operator 40 and provides the measured state(s) to the controller 20.

In some examples, the controller 20 is further in communication with at least one external sensor 36 configured to sense one or more parameters external to the vehicle 10.

The at least one external sensor 36 can include cameras, temperature sensors, ranging sensors, or any similar sensors configured to detect parameters external to the vehicle 10 or from which such parameters can be derived.

In some examples, the controller 20 is further in communication with one or more vehicle sensors 38. The one or more vehicle sensors 38 include sensors configured to detect one or more vehicle parameters (e.g. speed, acceleration, rotations per minute (RPMs), and the like).

In some examples, the controller 20 can further include a connection to a physiological sensing device 42 on the vehicle operator 40. The physiological sensing device 42 can include a smart watch, a fitness tracker, a phone or tablet including fitness tracking functionalities, and/or any similar physiological sensing device able to monitor one or more physiological metric of the operator 40 and able to communicate with the controller 20. The communication between the physiological sensing device 42 and the controller 20 can be via any wireless communication protocol. In one example, the communication is via a Bluetooth™ connection.

In some examples, the controller 20 includes a wireless communications system 26 able to communicate with one or more remote computing systems 50. The wireless communications system 26 can be a cellular network connection, a peer-to-peer (P2P) communication protocol, a cloud based internet connection, and/or any other wireless communication protocol.

Included with the controller 20 is an operator state module 22 and a driving condition module 24. The controller 20 uses a combination of data from the various sensors 36, 38 in communication with the controller 20 to classify a driver's current stress level state (e.g. highly stressed, moderately stressed, no stress) and to effectively identify opportunities to engage the vehicle operator 40 in one or more guided stress reduction exercises when the vehicle operator 40 has elevated stress levels.

In one example, the operator state module 22 utilizes images from the imaging sensor 30 to monitor gaze patterns of the operator 40 and a stress level is determined at least in part by applying established pattern recognition to the monitored gaze patterns. The pattern recognition may be established based on naturalistic data collection and experimentation from which differences between gaze distribution patterns at varying stress levels are established. The pattern recognition may be determined by empirical data gathering, application of machine learning systems to pre-established data sets, and/or any combination thereof.

The controller 20 uses the combined data from the sensors 30, 34, 36, 38 to intelligently determine when the vehicle 10 is in a conducive state (e.g., stopped, pulled over, etc.) for the operator 40 to perform the guided stress reduction exercise. A conducive state is any operative state of the vehicle 10 in which the vehicle operator 40 is able to reduce attention to the surrounding environment and focus on performance of the guided stress reduction exercise. In some examples, the vehicle 10 may still be operating, but have an expected conducive window at least long enough for the guided stress reduction exercise to be performed. One such example is while the vehicle 10 is stopped at a traffic light.

The guided stress reduction exercises can include any exercises known to relieve or reduce stress in an individual and can be provided from the controller 20 to an infotainment screen 60 in the vehicle 10. In one particular example, the guided stress reduction exercise includes a breathing exercise.

During the guided stress reduction exercise, the controller 20 uses the sensors 30, 34, 38, 42 to determine a quality of the operators performance of the guided exercise. The quality can be an unbounded numerical score, a rating within a range (e.g., a value of 1-10), a binary pass/fail, or any similar quality metric. The controller 20 then calculates a performance score of the operator 40 and reports the score to the operator 40. The performance score is based at least in part on the quality metric and may include other features such as a time from providing an alert to initiating the guided exercise. In some examples, this report can further include suggestions and recommendations for improving future performances of the exercise.

In yet further examples, the score can be gamified by the controller 20 providing rankings, relative scores, achievement metrics, high score tables, and the like. Gamification in this manner further incentivizes the vehicle operator 40 to improve their performance of the guided exercises. In some examples, the rankings and/or scores may be further tied to incentives, such as prizes, discount codes, etc., further encouraging the operator 40 to engage with the stress reduction exercises.

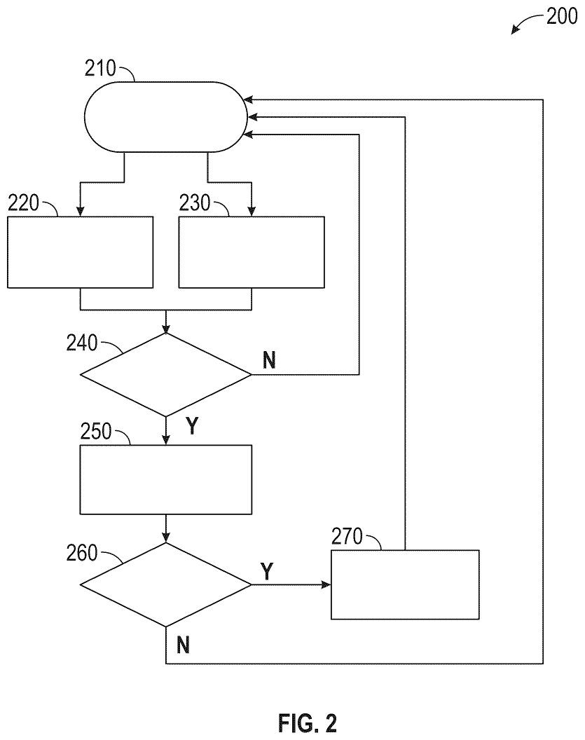

With continued reference to FIG. 1, FIG. 2 illustrates an example process 200 by which the controller 20 can initiate a guided stress reduction exercise. Initially, when the vehicle 10 is being operated, the process 200 continuously monitors the operator 40 of the vehicle 10, and the state of the vehicle 10 in a continuous monitoring step 210.

The monitoring step 210 simultaneously monitors a stress level condition 220 and a conduciveness condition 230.

The stress level condition 220 determines the stress level of the operator 40 based on gaze detection from the imaging sensor 30 and any available physiological data on the operator 40. In some examples, the stress level condition is evaluated using a machine learning process. In alternative examples, the stress level condition is evaluated using a rules based analysis. In yet further examples, the stress level condition is evaluated based on a combination of rules based analysis and machine learning.

The conduciveness condition 230 uses conventional monitoring systems, such as those utilized in automated vehicle operations and/or driver assistance systems, to determine a contextual environment in which the vehicle 10 is operating using the driving condition module 24. The driving condition module 24 analyzes the particular road conditions and environmental conditions alongside the current operational state of the vehicle 10 (e.g. stopped vs. moving) and determines when it is conducive to initiate a guided stress reduction exercise.

The conditions 220, 230 are provided to a check 240 and the check 240 determines if both conditions 220, 230 are met. Both conditions 220, 230 are met when the operator 40 is experiencing an elevated stress level (e.g., a stress level above a predetermined threshold) and the vehicle 10 is in a conducive state to engage in a guided stress reduction exercise.

When both conditions are not met, the process 200 returns to the continuous monitoring 210, and iterates.

When both conditions 220, 230 are met, the process 200 provides a notification to the operator 40 at a notification prompt step 250. The notification may be provided through an infotainment screen 60. In some examples, the notification may further include audio and visual alerts, haptic alerts through the physiological sensing device 42, or any other conventional alert style.

Once alerted, the operator 40 of the vehicle 10 is provided an opportunity to respond by approving the implementation of the guided stress reduction exercise in a driver approval check 260. The approval may be provided via any input means including pressing a button on the infotainment screen 60, providing an audible acknowledgement to or more microphones included in the vehicle, or any similar acknowledgment and acceptance.

If the operator 40 does not acknowledge and accept the guided stress reduction exercise, the process 200 resumes continuous monitoring at the continuous monitoring step 210, and may notify the operator 40 again once conditions have changed.

If the operator 40 does acknowledge and accept the guided stress reduction exercise notification, the process 200 initiates a guided stress reduction exercise via displaying instructions for performing the exercise on the infotainment screen 60 in an initiate session step 270. During performance of the guided stress reduction exercise, the operator 40 is monitored to determine how well the operator 40 performs the guided stress reduction exercise.

In one example, the guided stress reduction exercise is a guided slow breathing exercise, and the infotainment screen 60 displays a video demonstration of a vehicle operator engaging in the stress reduction exercise. In other examples, the guidance may be textually communicated steps, graphical illustrations of the exercise, or any other communication technique or combination of communication techniques.

In addition, while monitoring the performance of the operator 40, the controller 20 continues to monitor exterior conditions to ensure that the vehicle 10 remains in a conducive state. If the state is no longer conducive and/or when the guided stress reduction exercise completes the session is concluded and the process resumes the continuous monitoring step 210.

In some implementations, where the guided stress reduction exercise does not sufficiently lower the stress levels of the operator 40 and where the vehicle 10 is still in a conducive state upon conclusion of the guided stress reduction exercise, the process 200 may continue through the monitoring and return straight to the notification prompt 250.

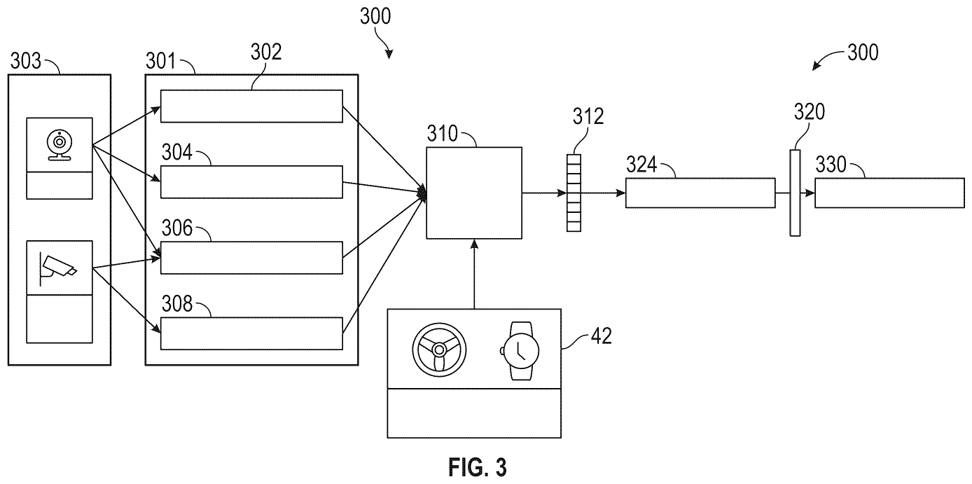

With continued reference to FIGS. 1-2, FIG. 3 illustrates an exemplary machine learning (ML) architecture 300 for determining a stress level of the operator 40. In the example architecture 300, sensors 303 (including sensors 30, 34, 42 illustrated in FIG. 1) provides multiple types of inputs 301 to a ML feature extractor and sub-task classification 310. The inputs 301 include emotional state inputs 302, eye gaze pattern inputs 304, body posture inputs 306 and operator action inputs 308. Each of the inputs 301 is part of a time series with each distinct measurement having a corresponding time of measurement.

The ML feature extractor and sub-task classification 310 uses the time of measurement to classify and organize the inputs into machine learning features. Each machine learning feature is a sequence of measurements and data from the inputs 301 for a given time t in the time series. The features are arranged in a standardized order allowing each feature to be directly compared to each other feature. In some examples, the features can be can be aggregated using a time smoothing process in order to eliminate or reduce the impact of outlier data points such as an isolated smile occurring during a high stress situation.

ML features 312 generated by the ML feature extractor and sub-task classification 310 are provided from the ML feature extractor and sub-task classification 310 to a noise reduction process 324 and then to an input layer of a Machine Learning (ML) system 320. The noise reduction processing 324 mitigates noise introduced into the inputs as a result of overlapping facial expressions (e.g. occasional smiles may occur even while an operator 40 is in a high stressed situation). Similarly, certain facial expressions may provide multiple distinct meanings depending on additional context (e.g., an open mouth occurring during a traffic jam may be indicative of high stress, whereas an open mount occurring while the driving conditions are good may not). In one example, the noise reduction processing 324 applies a time series smoothing process to the data.

The processed features are analyzed by the ML system 320 to determine a stress output 330. The stress output 330 is a numerical value (e.g. a scalar magnitude or a percentage) indicative of how elevated a stress level the operator 40 is currently experiencing. In one example, the stress output 330 sets a stress level of the operator 40 as one of highly stressed, moderately stressed, or not stressed.

The ML classification model that is employed by the ML classifier can be any conventional high-performing model that predicts a probability distribution of a set of emotions or direct stress metric(s) based on the sensing inputs. In some examples, the model is not stored within a memory of the controller 20. Instead, the model is retrieved from the remote computing systems 50 via the wireless communications 26 on an as needed basis.

In some examples, in order to minimize a loading time of the ML architecture 300, the model employed is a lightweight model. Lightweight ML models are models that prioritize efficiency and compactness while developing the machine learning model.

In some examples, the model generates predictions based only on the information provided in the most recently captured feature.

In some examples the model is a stateless model, with only a most recent output being stored between predictions.

In some examples, the sensors 303 providing the inputs 301 are sampled at a reduced frequency (e.g. once per second). Sampling the sensors 30, 34, 42 at the reduced frequency reduces the computing sessions required preform the ML analysis and reduces the computational load that the process 200 (illustrated in FIG. 2) imposes on the processor(s) of the controller 20.

In some examples, additional data from physiological sensors 42 may be further utilized by the ML feature extractor and sub-task classification 310 to validate the machine learning features.

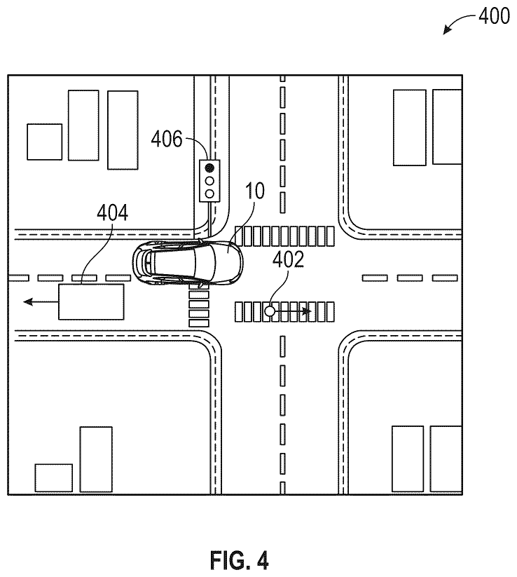

With continued reference to FIGS. 1-3, FIG. 4 illustrates an exemplary scene 400 in which the vehicle 10 is determined to be in a safe state for engaging in the guided stress reduction exercise. The scene refers to the environment through which the vehicle 10 traverses, as determined by the driving condition module 24 using data from the externally facing sensors 36. In the example of FIG. 4, the scene 400 includes a pedestrian 402 crossing parallel to a direction of travel of the vehicle 10, a second vehicle 404 traveling in an opposite direction of the vehicle 10 in a different lane than the vehicle 10, and a stop light 406. The scene is constructed from the external facing sensor data using any conventional scene construction. In some examples, the vehicle 10 may further enhance the scene 400 using data from a vehicle to vehicle (V2V) communication system, data from a vehicle to infrastructure (V2I) communication system, and/or a vehicle to anything (V2X) communication system.

In addition to the scene 400, the driving condition module 24 recieves data regarding the operations of the vehicle (e.g. speed, acceleration and direction) from the one or more vehicle sensors 38.

The scene 400 and the vehicle data are combined by the driving condition module 24 to generate a road context of the vehicle 10. Based on the road context derived, the driving condition module 24 identifies whether the vehicle is stopped, and if the vehicle 10 is stopped, identifies the context of the stoppage (e.g. stopped due to a traffic jam, or stopped due to a traffic light). The context can be derived based on established rules based metrics (e.g. when the vehicle is stopped and a traffic light is red, the stoppage is considered conducive), using a machine learning analysis that considers all sensor information, or a combination of the two.

Similar rules and analysis are employed by the driving condition module 24 to determine when a condition becomes unsafe during performance of a guided stress reduction exercise. When the stoppage is no longer conducive, the driving condition module 24 provides an interrupt to the guided stress reduction exercise via an alert ensuring that the operator 40 of the vehicle 10 is able to return to full attentiveness.

The terms “a” and “an” do not denote a limitation of quantity, but rather denote the presence of at least one of the referenced item. The term “or” means “and/or” unless clearly indicated otherwise by context. Reference throughout the specification to “an aspect”, means that a particular element (e.g., feature, structure, step, or characteristic) described in connection with the aspect is included in at least one aspect described herein, and may or may not be present in other aspects. In addition, it is to be understood that the described elements may be combined in any suitable manner in the various aspects.

When an element such as a layer, film, region, or substrate is referred to as being “on” another element, it can be directly on the other element or intervening elements may also be present. In contrast, when an element is referred to as being “directly on” another element, there are no intervening elements present.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this disclosure belongs.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof.

What is claimed is:

1. A vehicle comprising:
a first set of sensors configured to monitor an operator of the vehicle;
a second set of sensors configured to monitor a scene in which the vehicle is operating;
a controller including a driving condition module and an operator state module, the driving condition module being configured to determine a conduciveness for a guided stress reduction exercise using the second set of sensors and the operator state module being configured to determine a stress level of the operator based at least in part on the first set of sensors; and
the controller including a memory storing instructions for causing the controller to perform operations including:
identifying a stress level of the operator using the operator state module;
identifying a conduciveness of the scene to a guided stress reduction exercise using the driving condition module; and
initiating a guided stress reduction exercise in response to the stress level of the operator exceeding a threshold and the scene being conducive to performing a guided stress reduction exercise.

2. The vehicle of claim 1, wherein the guided stress reduction exercise is a guided breathing exercise.

3. The vehicle of claim 1, wherein the first set of sensors includes at least one camera defining a field of view including a face of the operator, and wherein the operator state module includes a gaze based driver monitoring system.

4. The vehicle of claim 3, wherein identifying the stress level of the operator comprises identifying a gaze pattern corresponding to an elevated stress level using the gaze based driver monitoring system.

5. The vehicle of claim 1, wherein the first set of sensors includes at least one physiological sensor disposed on the operator.

6. The vehicle of claim 5, wherein identifying the stress level of the operator includes correlating data in the first set of sensors as a set of time series machine learning features and providing the time series machine learning features to an input layer of a machine learning system, and wherein the machine learning system is configured to output a stress level of the operator.

7. The vehicle of claim 6, wherein the controller includes a wireless data connection, and wherein the machine learning system is retrieved from a remote data storage responsive to the controller initiating the operations.

8. The vehicle of claim 1, wherein the operations further include monitoring a performance of the guided stress reduction exercise by the operator and assigning a score to the performance.

9. The vehicle of claim 8, wherein the controller further includes and least one gamification feature configured to incentivize the operator increasing the score of the performance.

10. The vehicle of claim 1, wherein the operations further include interrupting a guided exercise in response to determining that the scene is no longer conducive to performance of the guided exercise.

11. A method for reducing stress in a vehicle operator comprising:
identifying, using an operator state module, a stress level of the operator based at least in part on a gaze pattern of an operator of a vehicle;
identifying a conduciveness to a guided stress reduction exercise of a scene in which a vehicle is operating using a driving condition module; and
initiating a guided stress reduction exercise in response to the stress level of the operator exceeding a threshold and the scene being conducive to performing a guided stress reduction exercise.

12. The method of claim 11, wherein initiating the guided stress reduction exercise comprises providing a notification to the operator indicating elevated stress levels and suggesting the guided stress reduction exercise.

13. The method of claim 12, wherein initiating the guided stress reduction exercise further comprises displaying the guided stress reduction exercise on an infotainment screen of the vehicle.

14. The method of claim 12, wherein initiating the guided stress reduction exercise further comprises monitoring a performance of the guided stress reduction exercise by the vehicle operator.

15. The method of claim 14, further comprising applying a score to the performance and ranking the performance using a gamification system.

16. The method of claim 11, wherein the operator state module includes a machine learning system configured to receive a set of inputs from a plurality of vehicle sensors including a driver monitoring camera, define machine learning features based on the set of inputs, and generate a stress level of the vehicle operator based on the defined machine learning features.

17. The method of claim 16, wherein the operator state module further includes a noise filtering process configured to filter the machine learning features using a time series smoothing process.

18. The method of claim 16, wherein the machine learning system a stateless model, with only a most recent output being stored between outputs.

19. The method of claim 11, further comprising receiving physiological data of the vehicle operator from at least one physiological sensor disposed on the vehicle operator, and wherein identifying the stress level of the vehicle operator is based at least in part on the physiological data.

20. The method of claim 11, wherein initiating guided stress reduction exercise further comprises continuously monitoring the conduciveness of engaging in the guided stress reduction exercise and responding to a change from conducive to non-conducive by interrupting the guided stress reduction exercise.
